SEQUENCE LISTING (20 sequences)

SEQ ID NO: 1 - DNA, 23 bp, Artificial Sequence
  Description of Artificial Sequence Synthetic primer
aacgccgtgagttcgtcgtatcg

SEQ ID NO: 2 - DNA, 28 bp, Artificial Sequence
  Description of Artificial Sequence Synthetic primer
catatagccgctttttctggtgtcttta

SEQ ID NO: 3 - DNA, 24 bp, Artificial Sequence
  Description of Artificial Sequence Synthetic primer
cctcacgccatcagtcccaaaaat

SEQ ID NO: 4 - DNA, 22 bp, Artificial Sequence
  Description of Artificial Sequence Synthetic primer
tgataggcaagcagacaacgaa

SEQ ID NO: 5 - DNA, 24 bp, Artificial Sequence
  Description of Artificial Sequence Synthetic primer
gatgtgtgtgtcaataccaccagc

SEQ ID NO: 6 - DNA, 22 bp, Artificial Sequence
  Description of Artificial Sequence Synthetic primer
gcaggacccacgctaagattgt

SEQ ID NO: 7 - DNA, 22 bp, Artificial Sequence
  Description of Artificial Sequence Synthetic primer
caggtcgccttcgcaatgagta

SEQ ID NO: 8 - DNA, 23 bp, Artificial Sequence
  Description of Artificial Sequence Synthetic primer
acgggaggcagcagtagggaata

SEQ ID NO: 9 - DNA, 20 bp, Artificial Sequence
  Description of Artificial Sequence Synthetic primer
gccgcgtggaggatgaaggt

SEQ ID NO: 10 - DNA, 24 bp, Artificial Sequence
  Description of Artificial Sequence Synthetic primer
tcatgcctttatcgccttttctcc

SEQ ID NO: 11 - DNA, 24 bp, Artificial Sequence
  Description of Artificial Sequence Synthetic primer
gtggaaaaaggcggtatcggtcaa

SEQ ID NO: 12 - DNA, 24 bp, Artificial Sequence
  Description of Artificial Sequence Synthetic primer
cgatcgctttgagaggtgcttttt

SEQ ID NO: 13 - DNA, 26 bp, Artificial Sequence
  Description of Artificial Sequence Synthetic primer
ctagagcctatgagtggggaatcttt

SEQ ID NO: 14 - DNA, 24 bp, Artificial Sequence
  Description of Artificial Sequence Synthetic primer
atccgttcccttaaccatagtgct

SEQ ID NO: 15 - DNA, 24 bp, Artificial Sequence
  Description of Artificial Sequence Synthetic primer
tgctaactaaccccccgctatggc

SEQ ID NO: 16 - DNA, 706 bp, Helicobacter pylori
catatagccgctttttctggtgtctttaccgattagaattttattcgtttgagaatgttttttaaaatacaatccggcagcaatgcctaaacgcatcacaaacatgggggtgagtttcacccctgctttacccctcacgccatcagtcccaaaaattttcatcgttataaaataccttttaaactatttttaatcaatttttagatagaattatgccaaattttacattacaaagggattaaaacaaggctatggcaaatcataagtccgcagaaaagcgaatcagacagaccattaagagaaccgaacgcaacaggttctataaaactaaaattaaaaatatcattaaagccgtgcgtgaagccgttgctgtcaatgatgtagcaaaagctcaagagcgtttgaaaatcgctaataaagagttgcataaatttgtcagcaaggggattttaaagaaaaacaccgcttctaggaaagtctcaaggcttaacgcttcagtgaaaaaaatcgctctcgcttagttttgtggcgttttcaacttctttaagctcagtaatgggtttttattattgggcttctttttaagttttgcgttttttagattgttgtattttttattcacatctttttataggtagtctcgcatgtccattctagccgaaaagctttcttccattctcaaacgatacgacgaactcacggcgtt

SEQ ID NO: 17 - DNA, 526 bp, Helicobacter pylori
gatgtgtgtgtcaataccaccagcagttacgatcaaaccttcaccggctaaggcttcagtagcaggacctacgctaagattgtttttaacgccatcttgcatgtctttgttaccgcctttaccaatgccagcgattttgccatctttaataccaatatccgctttataaataccggtgtaatccacgattaaagcgttagtgatgattagatccaattcttctttgctagggttgttggattggctcatgccttctctcagggttttaccgccaccgaatttaagctcttcgccataaatggtgtagtcatgttctacttcagcgatcaagtctgtatcgcccaatctcactttatcgcctgtagtagggccatacatagaaacatattcttttctgctaatctttttcatttcttactccttaattgtttttacatagttgtcatcgcttttagcgccatgaaaaccacgctctttagctctgtgtaaagcaatttttttgctttcgttgtctgcttgcctatca

SEQ ID NO: 18 - DNA, 370 bp, Helicobacter pylori
acgggaggcagcagtagggaatattgctcaatgggggaaaccctgaagcagcaacgccgcgtggaggatgaaggttttaggattgtaaactccttttgttagagaagataatgacggtatctaacgaataagcaccggctaactccgtgccagcagccgcggtaatacggagggtgcaagcgttactcggaatcactgggcgtaaagagcgcgtaggcgggatagtcagtcaggtgtgaaatcctatggcttaaccatagaactgcatttgaaactactattctagagtgtgggagaggtaggtggaattcttggtgtaggggtaaaatccgtagagatcaagaggaatactcattgcgaaggcgacctg

SEQ ID NO: 19 - DNA, 277 bp, Helicobacter pylori
gtagaaaaaggcggtattggtcaagtaactttccctatggtggctgatattaccaaaagcatttctagagactatgatgtgttgtttgaagaagcgatcgctttgagaggagcttttttgattgacaaaaacatgaaagtaaggcatgcggtgatcaatgacttaccattaggcagaaatgcagatgaaatgcttcgcatggtggacgctctcttacactttgaagaacatggtgaagtttgcccagcaggttggagaaaaggcgataaaggcatga

SEQ ID NO: 20 - DNA, 138 bp, Helicobacter pylori
atccgttcccttaaccatagtgctaactaaccccccgctatggcttgaatgggtggtttttaagaatttttcttgaatgtccaactcgctcaaatccatcgtaaaagaatccaaagattccccactcataggctctag

(19) United States
(12) Patent Application Publication
(10) Pub. No.: US 2006/0003350 A1
(43) Pub. Date: Jan. 5, 2006

(54) Methods and compositions to detect bacteria using multiplex PCR

(76) Inventors: Tat-Kin Tsang, Winnetka, IL (US); Xiangwen Meng, Skokie, IL (US); Hongjun Zhang, Glenivew, IL (US)

Correspondence Address: BARNES & THORNBURG, LLP, P.O. BOX 2786, CHICAGO, IL, 60690-2786, US

(21) Appl. No.: 11/114,361

(22) Filed: Apr. 26, 2005

Related U.S. Application Data
(63) Continuation-in-part of application No. 10/834,151, filed on Apr. 28, 2004.

(51) Int. Cl. C12Q 1/68; C12P 19/34

(52) U.S. Cl. 435/6; 435/91.2

(57) ABSTRACT

A novel multiplex polymerase chain reaction assay for detecting a bacteria (e.g. Helicobacter pylori) in a specimen uses multiple oligonucleotide primer pairs based on the sequences of multiple loci of the bacteria. In one application, up to five loci in the genomic DNA sequences of Helicobacter pylori were amplified. Two fragments of H. pylori were amplified from each locus, wherein a second fragment was an internal fragment of the first fragment.

This application is a continuation in part of copending U.S. Ser. No. 10/834,15 filed on Apr. 28, 2004.

A one-step multiplex PCR (polymerase chain reaction) improves diagnostic accuracy for detection of Helicobacter pylori.

### BACKGROUND

Microorganisms such as bacteria and viruses cause serious infectious diseases such as tuberculosis, cholera, hepatitis and meningitis. To diagnose and cure bacterial infections requires rapid and early identification of specific disease causing pathogens in clinical specimens. Several bacterial infections do not show characteristic symptoms initially, which requires sensitive and specific tools to diagnose infections.

Helicobacter pylori infection is one of the most common bacterial infections in humans. The prevalence of H. pylori infection is worldwide and may be as high as 80% in developing countries and up to 40% in developed countries. However, the mode of transmission, the natural history, and other aspects of the epidemiology of H. pylori infection are still unclear. Presence of Helicobacter pylori bacteria in gastric mucosa and epithelia may be associated with chronic gastritis, peptic and duodenal ulcers, and gastric cancers.

Detection of H. pylori is generally accomplished in two ways: (1) directly, by examining a stomach biopsy by histology, cell culture, and other methods to analyze tissue specimens; or (2) indirectly, by testing a sample of peripheral blood serum for circulating antibodies against H. pylori. Some methods require endoscopy with a biopsy. Direct detection methods currently available for H. pylori, include bacterial culture, histology, serology, stool antigen test, rapid urease test (Campylobacter-like organism or CLOtest), isotope urease breath test, and conventional polymerase chain reaction (PCR).

The PCR based methods are sensitive and specific when compared to the urease assay in the presence of other urease-positive bacteria. PCR based methods are relatively inexpensive and produce results faster than bacterial culture, because culturing H. pylori is lengthy and is not possible in many situations. Conventional PCR generally uses a set of primers to amplify a genetic region in H. pylori. PCR tests for various bacteria, including H. pylori, are available for a broad spectrum of specimens, including laboratory cultures and clinical samples. Although conventional PCR based detection methods have many advantages, there are some disadvantages due to both frequent false-positive and false-negative results. The presence of polymorphisms at the primers' binding sites may lead to low specificity in conventional PCR tests.

### SUMMARY OF THE DISCLOSURE

A novel one-step multiplex polymerase chain reaction detection system using a plurality of genetic loci was developed to identify a pathogenic bacteria present in clinical specimens. In one application, up to five loci in the genomic DNA sequences of Helicobacter pylori were chosen as amplification targets. Two fragments of H. pylori were amplified from each locus, wherein a second fragment was an internal fragment of the first fragment.

A method of detecting a bacteria in a specimen includes the steps of:
(a) performing a multiplex polymerase chain reaction, wherein a plurality of DNA fragments representing a plurality of loci in the bacteria are amplified by a plurality of primers, where for each pair of forward and reverse primers, one primer is internal to the other; and
(b) determining that a specimen is positive for the bacteria if a number of amplified fragments is sufficient to detect the bacteria from other bacteria.

The primers used to amplify multiple loci in a bacteria include the following characteristics:
(a) are about twenty to thirty bases long;
(b) have a melting temperature of about 60° C.;
(c) have a GC content of about fifty percent;
(d) have minimal dimer formation; and
(e) have low frequency of mutations in the primer binding site.

The multiplex polymerase chain reaction is performed with an isolated bacterial DNA, wherein the bacterial DNA is isolated from a clinical sample. The clinical sample is selected from a group of gastrointestinal tract tissue, stool, urine, blood, saliva, mucus secretions, dental plaque, and other tissues capable of containing the bacteria. The multiplex polymerase chain reaction is also suitably performed directly on a biological sample or a specimen.

A diagnostic kit to detect a pathogenic bacteria in a specimen includes in discrete containers:
(a) a plurality of primers to amplify a plurality of DNA fragments from the bacterial genomic sequence; and
(b) reagents to perform a multiplex polymerase chain reaction.

The diagnostic kit to detect the bacteria also includes a DNA polymerase, nucleotides, and buffers. The plurality of primers in the diagnostic kit are capable of amplifying a plurality of DNA fragments so that half of the amplified fragments are internal to the other half of the amplified fragments.

A method for detecting Helicobacter pylori in a specimen includes the steps of:
(a) performing a multiplex polymerase chain reaction, wherein a first set of primers amplifies a first set of DNA fragments from a Helicobacter pylori genomic sequence and a second set of primers amplifies a second set of DNA fragments from the Helicobacter pylori genomic sequence that are internal to the first set of DNA fragments; and
(b) determining that a specimen is positive for Helicobacter pylori if at least fifty percent of all the fragments or at least two fragments representing either the first set or the second set of DNA fragments and the corresponding internal fragments are amplified.

A method for detecting Helicobacter pylori in a specimen includes the steps of:
(a) performing a multiplex polymerase chain reaction, wherein
   (i) a first DNA fragment from a Helicobacter pylori genomic sequence is amplified by a first primer pair and an internal segment of the first DNA fragment is amplified by a second primer pair,
   (ii) a second DNA fragment from a Helicobacter pylori genomic sequence is amplified by a third primer pair and an internal segment of the second DNA fragment is amplified by a fourth primer pair; and
(b) determining that a specimen is positive for Helicobacter pylori if at least one of the fragments and its internal fragment or fifty percent of all the fragments are amplified.

A multiplex polymerase chain reaction with the first set of primers was performed separately from the multiplex polymerase chain reaction with the second set of primers. The first set of primers and the second set of primers amplified a total of ten fragments representing five loci in the Helicobacter pylori sequence.

The Helicobacter pylori locus is selected from a group of coding, non-coding, exons, introns, and regulatory regions. A plurality of loci to be amplified was selected from a group of DNA sequences that include a 0.86 kb DNA fragment, Urea A gene, 16S rRNA, a DNA sequence encoding a 26 kDa antigen, Hpa A gene, whose nucleotide sequences are listed in FIG. 7 and CagA, UreaC, and Flagellin of H. pylori, whose sequences are known and available. A plurality of primers used to amplify the loci was selected from a group of primers whose DNA sequences are listed in TABLE 2. The primer pairs used to amplify multiple loci in H. pylori are designed or derived from a group of DNA sequences comprising a 0.86 kb DNA fragment, Urea A gene, 16S rRNA, a DNA sequence encoding a 26 kDa antigen, and Hpa A gene, whose nucleotide sequences are listed in FIG. 7. Primer sequences are derived or obtained from the target loci.

In a multiplex PCR disclosed herein, the first primer pair and the second primer pair have a common primer per locus. The primer pairs were selected from the primers listed in TABLE 2. A multiplex polymerase chain reaction to detect Helicobacter pylori was performed, wherein up to ten DNA fragments representing five H. pylori loci were amplified by fifteen primers, the ten DNA fragments representing five internal fragments. A set of fifteen primers comprise five forward and ten reverse primers.

A method of detecting Helicobacter pylori in a specimen includes the steps of:
(a) designing a plurality of primers in a plurality loci of Helicobacter pylori, wherein the primers
   (i) are specific for Helicobacter pylori;
   (ii) have a low frequency of mutations in primer binding sites;
(b) performing a multiplex polymerase chain reaction wherein the plurality of primers amplify a plurality of DNA fragments representing the plurality of loci in Helicobacter pylori; and
(c) determining that a specimen is positive for Helicobacter pylori if at least fifty percent of all the DNA fragments or at least four DNA fragments representing two loci in Helicobacter pylori are amplified or by other empirically or statistically derived criteria.

A diagnostic kit to detect Helicobacter pylori in a specimen includes in discrete containers:
(c) a first set of primers to amplify a first set of DNA fragments from a Helicobacter pylori genomic sequence;
(d) a second set of primers to amplify a second set of DNA fragments from Helicobacter pylori genomic sequence that are internal to the first set of DNA fragments; and
(e) reagents to perform a multiplex polymerase chain reaction.

The diagnostic kit to detect Helicobacter pylori also includes a DNA polymerase, deoxynucleotides, and buffers. The kit includes a set of primers whose nucleotide sequences are listed in TABLE 2. The kit may also include reagents: a buffer comprising 100 mM Tris-HCl (pH 8.3), 500 mM KCl, 16 mM MgCl₂, 0.01% (weight/volume) gelatin; and 10 mM of each deoxynuclotide. The concentration of primers is about 1.0 μM. A multiplex polymerase chain reaction is performed in a single reaction chamber, e.g., a single tube, in particular when in a kit.

A method for detecting Helicobacter pylori in a specimen includes the steps of:
(c) performing a multiplex polymerase chain reaction, wherein a plurality of primers amplify a plurality of Helicobacter pylori nucleic acid fragments are selected from the group consisting of nucleic acid molecules whose sequences are designated 0.86 kb DNA fragment, Urea A gene, 16S rRNA, a DNA sequence encoding a 26 kDa antigen, Hpa A gene, CagA, UreaC, and Flagellin; and
(d) determining that a specimen is positive for Helicobacter pylori if a number of amplified fragments is sufficient to detect the bacteria in the specimen.

A plurality of nucleic acid fragments comprise a subset of a plurality of internal fragments. The plurality of Helicobacter pylori nucleic acid fragments is selected from the group that includes RNA, cDNA, and genomic DNA.

Definitions

Locus: location of a DNA sequence in a chromosome that encodes one or more products.

Multiplex PCR: A variant of conventional polymerase chain reaction that uses at least two or more primer pairs to amplify different stretches of a target DNA molecule simultaneously.

Nested PCR: A modified polymerase chain reaction that uses one or more primers (“nested”) whose sequences are complementary to an internal site of a DNA molecule that has been amplified with other primers.

Nucleic acid: includes DNA, RNA, cDNA, genomic DNA, natural, synthetic, nucleic acid analogs, and the like.

Oligonucleotide: single stranded DNA molecule with any length ranging from four to about 100 nucleotides.

Primers: Oligonucleotides of about 6 bp to about 50 bp in length used for initiating polymerase chain reaction.
Forward: a primer that may bind to one of the two complementary anti-parallel DNA strands.
Reverse: primer that may bind to a strand that is complementary to the strand to which the forward primer binds.

Specimen: A biological sample such as saliva, stools, urine, blood, gastric biopsy, gastrointestinal tissue, tumor cells, mucus secretions, dental plaque, and other biological tissues, meat products, food products, and environmental samples such as soil, water.

### BRIEF DESCRIPTION OF THE DRAWINGS

FIG. 1 is an illustration of primer design for each locus (A-E) in H. pylori. FC stands for forward primer, which is used as a common primer. R1 and R2 represent two reverse primers. Amplicons FC-R1 and FC-R2 represent two fragments amplified with FC and R1, FC and R2, respectively. Between the two amplified DNA fragments, one fragment is internal to the other fragment. An image of the agarose gel electrophoresis stained with ethidium bromide shows the FC-R1 and FC-R2 amplified fragments in the selected loci (A-E).

FIG. 2 shows images of agarose gel electrophoresis by which H. pylori is detected by a multiplex PCR method. An image of the agarose gel electrophoresis stained with ethidium bromide shows DNA bands amplified using A5 and B5 set of primers is shown in (A) and amplified using M5 set of primers is shown in (B) from various H. pylori strains (ATCC Nos. 43504, 26695, and J99: ATCC 700824). M10 in (A) refers to the lanes containing 10 amplified bands. M in both (A) and (B) refer to a DNA standard. A5/B5/M5 refer to lanes containing 5 DNA bands that correspond to the 5 different loci. A5/M5-1 contains 5 FC and 5 R1 primers. B5/M5-2 contains 5 FC and 5 R2 primers. M10 contains 10 primer pairs. Each locus has two bands, but in different lanes except in lane M10.

FIG. 3 is an image of the agarose gel electrophoresis stained with ethidium bromide showing the sensitivity of H. pylori multiplex PCR amplification system. M refers to a lane containing DNA standard and pg refers to picograms of DNA used for amplification.

FIG. 4 is a gel picture showing the specificity of H. pylori multiplex PCR amplification system. S1-S5 refer to the various samples used. S1-E. Coli, S2-Enterobacter aerogenes, S3-Enterobacter cloacae, S4-Enterococcus spp, S5-Viridans group, J99-H. pylori (ATCC 700824). The arrows indicate the presence of the 16S rRNA.

FIG. 5 shows the detection of H. pylori by multiplex PCR from clinical samples S1-S6 (A) and from clinical samples S7-S12 (B). M refers to a lane containing a DNA standard.

FIG. 6 is an illustration of a single primer pair PCR and a multiplex PCR (in one tube and two tubes). The illustration represents amplification from 5 different loci of H. pylori.

FIG. 7 shows the DNA sequences of five different loci from H. pylori that were selected for multiplex PCR.

FIG. 8 Immunohistochemistry Stained Gastric Mucosa Demonstrating Helicobacter pylori, 1000× Magnification.

FIG. 9 Visual Analog Scale for Grading Inflammatory Activity. Adapted from M F Dixon et al. Am J Surg Pathol 20(10): 1996.

FIG. 10 shows primers selected for each locus. A, B, C, D, and E are five selected DNA loci of H. pylori. Each locus has two bands but in different lanes. The lower band is the same as nested PCR product of the larger band in each locus.

### DETAILED DESCRIPTION OF THE DISCLOSURE

A plurality of DNA fragments representing a plurality of loci from a bacterial genome is amplified using nested primers in a multiplex PCR reaction to positively identify the bacterial species. A plurality of DNA fragments from a specimen containing bacteria is amplified by a plurality of primers such that half of all the amplified fragments are internal to the other half.

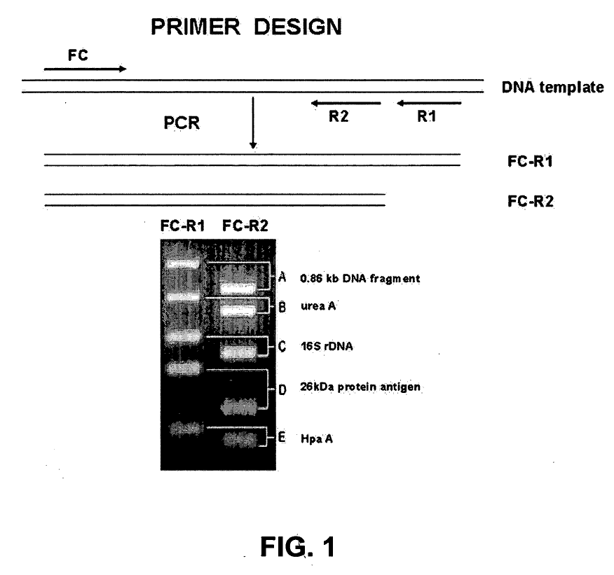

For each bacterial locus, one forward primer is selected as a common primer (FC) and two reverse primers (R1 and R2) are selected, in which R2 is inside the amplifying region of R1 (FIG. 1). In addition, these primers have the following characteristics: Tₘ, of about 60° C.; G+C: A+T, about 50%; length, 10-30 nt; minimal dimer formations with other primers; fewer mutations in the primer binding site; and are specific for a particular bacterial species.

In one example, up to five genetic loci from a bacterial genome are amplified using a set of at least five forward primers and ten reverse primers such that five of the amplified fragments are internal to the other five amplified fragments. A specimen is considered positive if at least five out of the ten amplicons are amplified or at least two of the five loci have both of their amplicons amplified. The amplified fragments can be resolved in a standard agarose gel electrophoresis or can be quantified using any other techniques such as real time quantitative PCR.

The number of loci to be selected for amplification depends on sequence similarity of a target bacterial pathogen to other commonly occurring bacteria, availability of non-conserved genomic regions in the target bacteria when compared to the commonly occurring bacteria, availability of conserved genomic regions among the various strains of the target bacterial pathogen, and technical and practical feasibility to accommodate multiple samples.

Two or more genetic loci from a bacterial genome are amplified using a set of at least two forward primers and four reverse primers such that two of the amplified fragments are internal to the other two amplified fragments.

In one example, a multiplex PCR, wherein five forward primers and five reverse primers representing five different bacterial loci are mixed to react in one reaction system and the five forward primers and five nested primers are allowed to react separately in another system. Alternatively, the five forward primers and the ten reverse primers are allowed to react in a single reaction system.

A multiplex PCR amplification was performed with isolated bacterial DNA from clinical samples that included infected tissues. A multiplex PCR amplification was performed with isolated bacterial DNA from bacterial cell cultures.

Amplifying more than one region of a nucleic acid molecule at the same time overcomes false-negative results because the possibility to amplify all or some of the selected DNA region is considerably higher when multiple regions are used rather than a single region. The amplified internal DNA fragments are helpful in minimizing false-positives. False negatives can be picked up by the one-step multiple-nested PCR. Unless the entire selected loci scanned by the multiplex PCR are deleted or mutated, the presence of some amplified fragments acts as an internal control, suggesting that the reaction has not failed, and helping rule out a false-negative result.

A standard touchdown PCR program was employed to amplify the bacterial DNA fragments. Briefly, a touchdown PCR involves decreasing the annealing temperature by 1° C. every second cycle to a ‘touchdown’ annealing temperature, which is then used for about 10 cycles, to optimize annealing temperatures. The basic idea is that any differences in Tₘ between correct and incorrect annealing gives a 2-fold difference in product amount per cycle (4-fold per ° C.). Therefore, the correct product is enriched over any incorrect products.

A diagnostic kit to detect bacteria includes in discrete containers, primers, whose nucleotide sequences are determined based on the criteria described herein, a suitable DNA polymerase such as, for example, Immolase™ (Bioline, London, UK), deoxynucleotides, buffers, and optionally a set of pre-amplified bacterial DNA fragments for comparison, and a control bacterial DNA.

A diagnostic kit to detect bacteria includes a set of up to five forward primers representing up to five different bacterial loci and up to ten reverse primers (including five nested primers) representing the five loci.

Multiplex PCR was used to amplify a plurality of DNA fragments from a plurality of loci at the same time to identify Helicobacter pylori. Improved PCR sensitivity and specificity are due to selected amplification of various nested DNA regions. An internal control for each amplicon enhances identification of false negatives because amplification of some fragments indicates that the multiplex reaction has not failed.

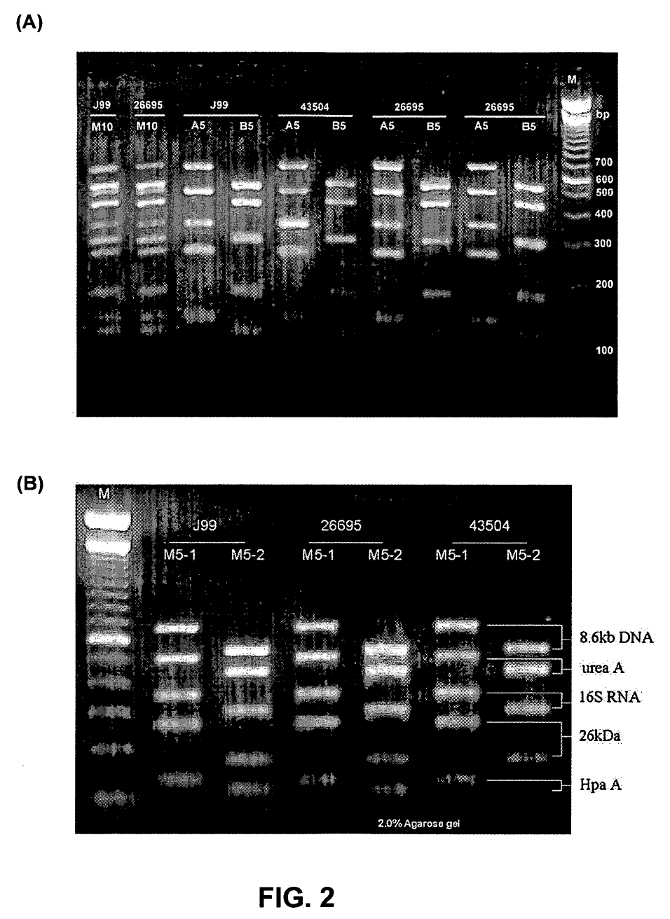

In an example of a one-step multiplex PCR detection system disclosed herein, five loci (Hpa A gene, 26 kDa protein antigen, 16S ribosomal RNA, urease A gene and a specific 0.86 kb DNA fragment) in Helicobacter pylori's genome were chosen as amplification targets. For each locus, one forward primer (FC) and two reverse primers (R1 and R2) were designed such that one amplified fragment is internal to the other amplified fragment. For example, amplicon FC-R2 is internal to amplicon FC-R1 (FIG. 1). After 32-40 cycles of amplification using Helicobacter pylori (strains J99, ATCC No. 26695 and ATCC No. 43504) DNA as templates, 10 DNA fragments were obtained with the multiplex PCR detection system. The amplified fragments were displayed in a 2% agarose gel with ethidium bromide staining (FIG. 2). The amplified fragments correspond to the targets as follows: 0.86 kb DNA fragment, 706 bp and 574 bp; Urea A gene, 526 bp and 465 bp; 16S rRNA, 371 bp and 315 bp; 26 kDa, 277 bp and 183 bp; Hpa A gene, 138 bp and 118 bp. For direct detection of H. pylori from a bacterial culture, a colony was picked with a pipette tip and diluted with Tryptic Soy Broth or TE, then used as template to perform amplification directly. The ten expected DNA fragments were directly amplified from the cultures of the three standard H. pylori strains.

Novel multiplex PCR primers were designed by reviewing several published primers that are specific for H. pylori genes and searching the GenBank, urease genes (ureA), 26 kDa protein antigen, Hpa A gene, 0.86 kb DNA fragment, and DNA sequences of 16S ribosomal RNA as amplification targets.

The 0.86 kb locus (706 bp) includes ureC and prfA genes. ureC encodes an urease protein involved in central intermediary metabolism and prfA gene encodes a peptide chain release factor RF-1 involved in protein synthesis. The ureA locus (526 bp) encodes an urease protein involved in central intermediary metabolism and responsible for urease activity. The 16S rRNA locus (370 bp) is a gene encoding 16S ribosomal RNA. The 26 kDa locus (277 bp) includes tsaA gene that encodes alkyl hydroperoxide reductase involved in cellular processes such as detoxification. The hpA locus (138 bp) includes hpaA gene that encodes a flagellar sheath adhesin involved in cellular processes such as chemotaxis and motility. An exemplary genomic sequence of H. pylori 26695 can be acquired from GenBank database with accession number AE000511.1. Nucleotide sequences for flagellin A (flaA; HP0601; GeneID: 899977), flagellin B (flaB; HP0115; GeneID: 900158), flagellin B (H. pylori J99; jhp0107; GeneID: 889505), cagA (cag island protein, CYTOTOXICITY ASSOCIATED IMMUNODOMINANT ANTIGEN; H. pylori J99; jhp0495; GeneID: 889201), and urease protein (ureC; HP0075; GeneID: 900169) can be obtained from the GenBank using the GeneIDs mentioned above.

For each H. pylori locus, a forward primer was selected as the common primer (FC) and two reversed primers (R1 and R2) were selected, in which R2 is inside the amplifying region of R1 (FIG. 1). In addition, these primers met the following criteria:
(b) Tₘ, around 60° C.
(c) G+C: A+T, about 50%;
(d) length, 10-30 nt;
(e) minimal dimer formations with other primers;
(f) fewer mutations in the primer binding site after checking the published H. pylori DNA sequences, and
(g) were specific for H. pylori.

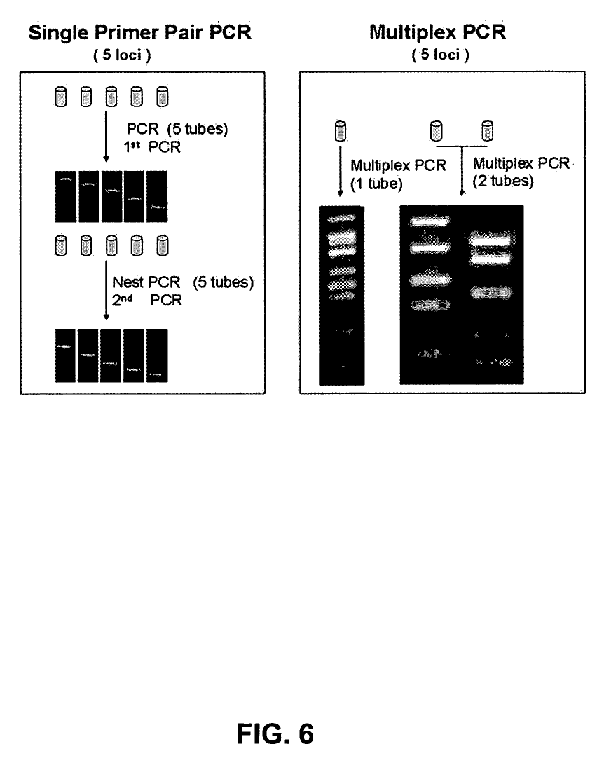

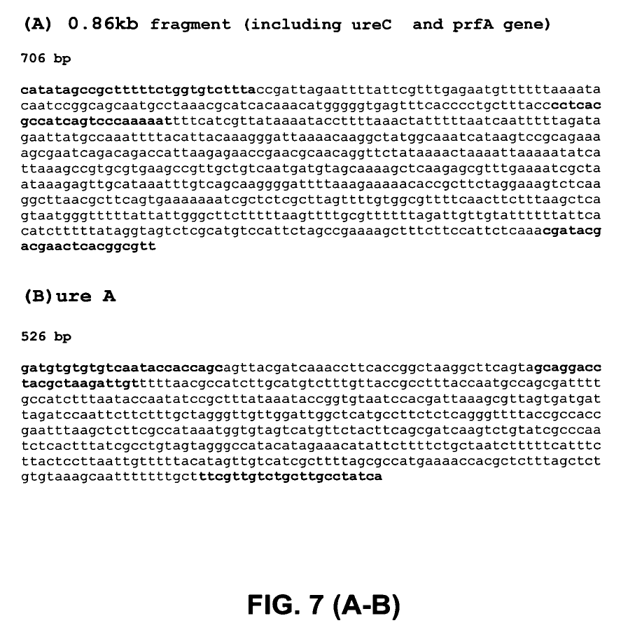

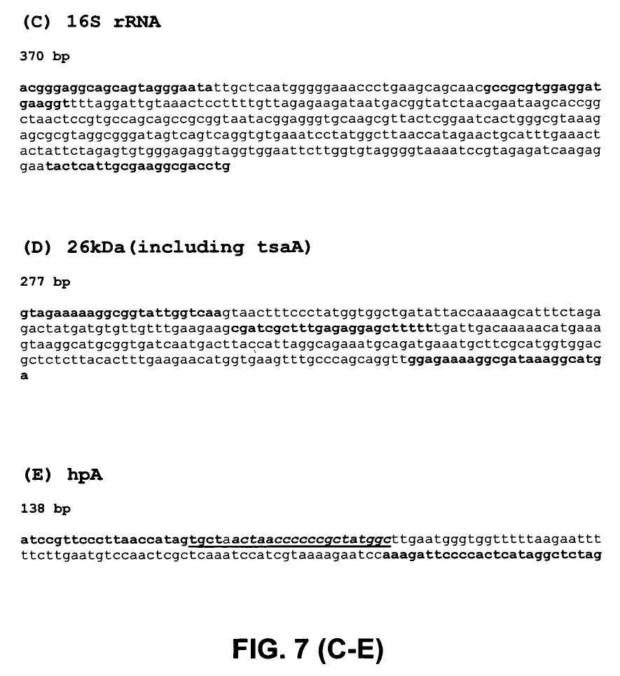

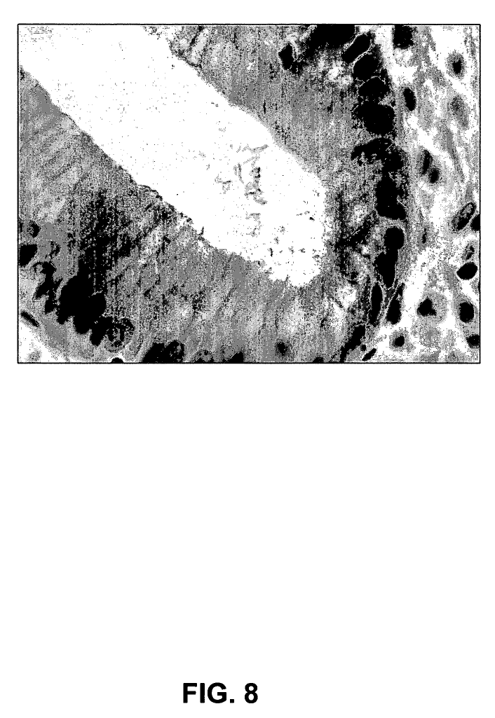

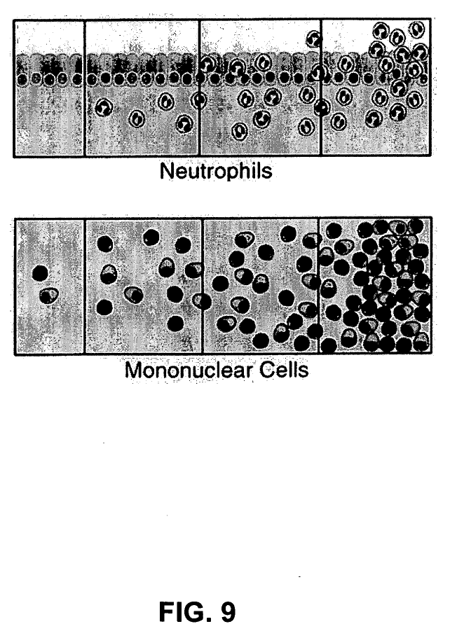

To balance the primer mixture, in an embodiment, all primers were divided partitioned in two ways: a system in which all the primers were mixed together (system 1), and system 2 where five forward primers were mixed with either one set of reverse primers (system 2) separately (FIG. 6). System 1 amplified 10 DNA fragments in each tube at the same time, wherein system 2 amplified 5 DNA fragments in each tube.

Two or more genetic loci from H. pylori genome were amplified using a set of at least two forward primers and four reverse primers such that two of the amplified fragments are internal to the other two amplified fragments. For example, for the Ure A gene loci of H. pylori, a forward primer and two reverse primers are used to amplify a 526 bp and a 465 bp fragments respectively (TABLE 2). For the Hpa A gene loci, a forward primer and two reverse primers are used to amplify a 138 bp and a 118 bp fragments respectively (TABLE 2). A specimen is considered positive for H. pylori if at least the 526 bp fragment and its internal 465 bp fragment is present (in the case of Urea A gene) or the 138 bp fragment and its internal 118 bp fragment is present (in the case of Hpa A gene) or at least one fragment from the Urea A gene loci and at least one fragment from the Hpa A gene loci are present.

Up to five genetic loci from H. pylori genome were amplified using a set of at least five forward primers and ten reverse primers such that five of the amplified fragments are internal to the other five amplified fragments (TABLE 2). A specimen is considered positive for H. pylori if at least five out of the ten amplicons illustrated in TABLE 2 are amplified or at least two of the five loci have both of their amplicons amplified (for example, the 526 bp fragment and its internal 465 bp of the Urea A gene and the 138 bp fragment and its internal 118 bp fragment of the Hpa A gene).

A multiplex PCR, wherein the five forward primers and the five reverse primers (R1-R5) were mixed to react in one reaction system and the five forward primers and the five nested primers (RN1-RN5) were allowed to react separately in another system (TABLE 2 and FIG. 6).

A multiplex PCR amplification was performed with isolated H. pylori DNA from clinical samples that included gastric biopsies and from bacterial cell cultures.

A diagnostic kit to detect H. pylori includes in discrete containers, primers, whose nucleotide sequences are described in TABLE 2, a suitable DNA polymerase such as, for example, Immolase™ (Bioline, London, UK), deoxynucleotides, buffers, and optionally a set of pre-amplified H. pylori fragments for comparison, and a control H. pylori DNA. The diagnostic kit may also include a set of up to five forward primers representing five different H. pylori loci and 10 reverse primers (including 5 nested primers) representing the five loci.

In one aspect, the diagnostic kit to detect H. pylori includes a set of at least two forward primers representing two different H. pylori loci and four reverse primers (including 2 nested primers) representing the two loci.

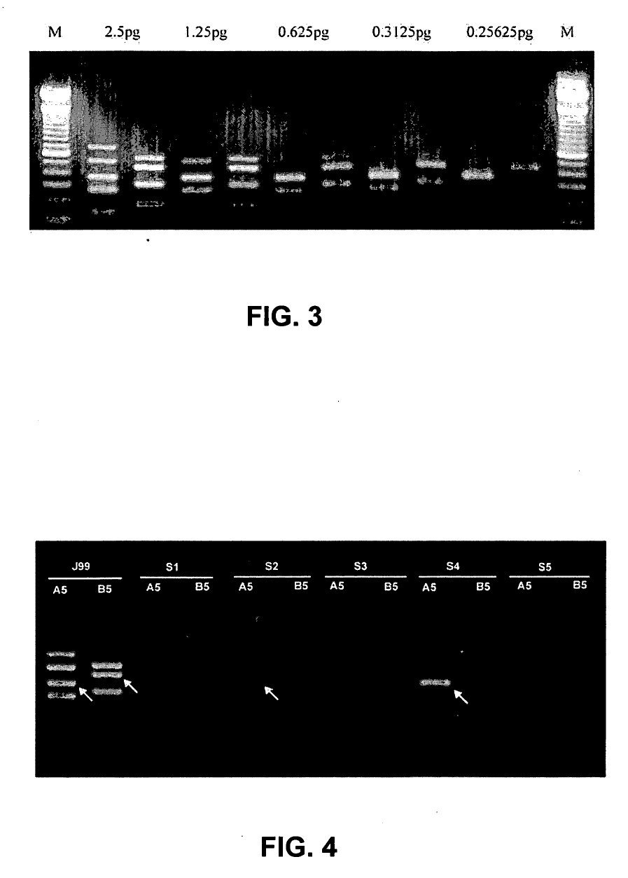

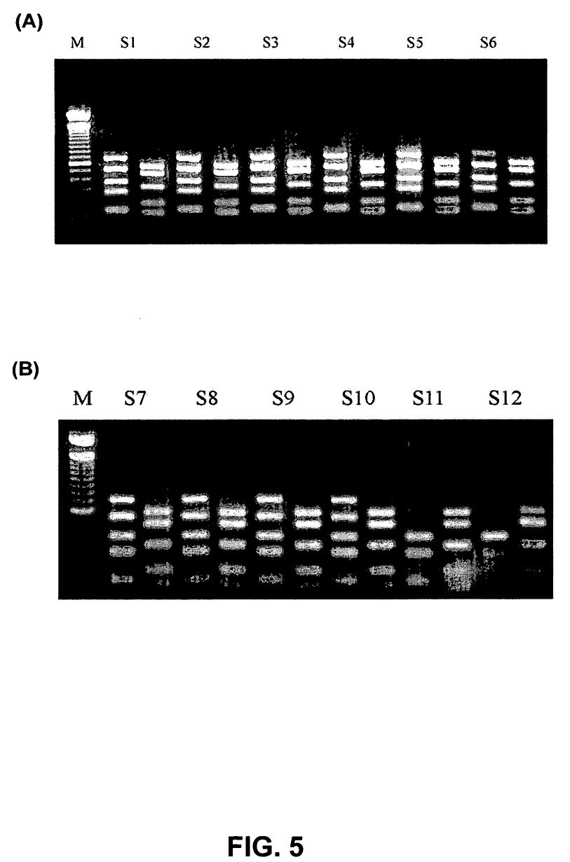

The sensitivity of the one-step multiple-nested PCR assay (multiplex PCR) was investigated through a 36-40 cycle amplification of different dilutions of H. pylori DNA. The multiplex PCR method disclosed herein, was able to detect 10 DNA bands (in two tubes) in a single reaction when the DNA of H. pylori from the template was diluted to as little as 2.5 pg (FIG. 3).

The specificity of the one-step multiple-nested PCR assay was investigated with 11 bacterial species, using H. pylori J99 as a positive control. None of the sets of fragments amplified from the 11 bacterial species displayed the standard H. pylori J99 multiplex PCR band pattern and only 2 bacterial species Enterobacter aerogenes and Enterococcus Spp., showed one 16S rRNA band.

The internal controls of the one-step multiple-nested PCR to detect H. pylori can minimize false-positives caused by homologous DNA sequences among various species in the primer binding sites. For example, one 16S rRNA band was generated for Enterobacter aerogenes and Enterococcus Spp. They can be distinguished from H. pylori, however, because both fragments from one locus and multiple loci are amplified in the case of H. pylori and not with the other bacterial species that were tested.

Amplified DNA fragments from a specimen containing H. pylori are confirmed with the one-step multiple-nested PCR because the disclosed primers produce two fragments for each of the five loci, one internal to the other. When both fragments are present for each locus, the DNA sample is inferred to be from H. pylori. On the contrary, if only one band is present for each locus, it may be caused by polymorphism in the amplified region.

Based on a proposal made by Monteiro et al. (2001), that a traditional PCR diagnosis for H. pylori is considered positive when one of two biopsied specimens from each part of the stomach is positive using two sets of primers derived from different genes, the disclosed one-step multiple-nested PCR assay was considered positive for H. pylori if 5/10 fragments or both DNA fragments from 2/5 loci were amplified.

To compare the sensitivity and the specificity of the multiplex PCR assays disclosed herein to some of the standard H. pylori tests, a comparative experimental analysis was performed. The results of multiplex PCR assays for H. pylori and the standard CLOtest and histological method shown in TABLE 1, indicate that the multiplex PCR system described herein is a sensitive and a specific method for identifying H. pylori in a clinical specimen.

### EXAMPLES

### Example 1

### Comparison of Multiplex PCR with CLOtest for Detection of H. pylori

Ninety patients with dyspepsia symptoms undergoing endoscopy were selected. To overcome the problem of patchy H. pylori distribution so that the organism is not captured in the tested tissue sample, the same gastric specimen was used for both the CLOtest and PCR assay. The CLOtest was performed first following standard protocols and the results were obtained from about 20 minutes to 24 hours. The specimens were collected from the CLOtest gel, DNA was isolated, and the one-step multiplex PCR was performed.

The multiplex PCR amplification was performed for about 36-40 cycles and the amplified PCR products were detected on an agarose gel electrophoresis system. Using the standard Helicobacter pylori DNA as a template, 10 expected DNA fragments were obtained after amplification with the PCR method disclosed herein. On a 2% agarose gel, the following DNA fragments were displayed like two DNA ladders: a) 706 bp, 526 bp, 371 bp, 277 bp, 138 bp and b)574 bp, 465 bp, 315 bp, 183 bp, 118 bp. For the multiplex PCR analysis, a specimen was considered positive for H. pylori if 5 out of 10 DNA fragments or both DNA fragments from 2 out of the 5 loci were amplified.

Positive results were obtained in 68% (61/90) with multiplex PCR and 43% (39/90) with CLOtest (TABLE 1). The sensitivity and specificity was 62% and 97% for CLOtest relative to multiplex PCR respectively (TABLE 1). The 10-primer pair-combination plus the internal control of each locus showed the multiplex PCR assay to be a rapid, sensitive and uniquely specific method for identification of Helicobacter pylori from various sources.

### Example 2

### Comparison of Multiplex PCR with Histology for Detection of H. pylori

Four to five gastric specimens were collected from 90 patients with dyspepsia symptoms undergoing endoscopy. One specimen was sent to the clinical laboratory for the histology study performed by an expert clinical pathologist. The other two or three specimens were collected for DNA extraction and the one-step multiplex PCR was performed as described in Example 1. For the multiplex PCR analysis, a specimen was considered positive for H. pylori if 5 out of 10 DNA fragments or both DNA fragments from 2 out of the 5 loci were amplified.

As disclosed in Example 1, positive results were obtained in 68% (61/90) with multiplex PCR and 11% (10/90) with histology study (TABLE 1). The sensitivity and specificity was 16% and 100% for the histology method relative to multiplex PCR respectively (TABLE 1). The 10-primer pair-combination plus the internal control of each locus showed the multiplex PCR assay to be a rapid, sensitive and uniquely specific method for identification of Helicobacter pylori from various sources. Although false negative results may be obtained in the samples used for histology because the patchy H. pylori distribution present results still indicated that the disclosed multiplex PCR method is a highly specific and sensitive valuable method in the detection of H. pylori when an invasive diagnostic is justified.

### Example 3

### Food Products Tests for H. pylori

Eleven fresh raw chickens and 18 orders of ready-to-eat raw tuna meat were collected from local grocery and restaurants respectively. The samples were washed with enough volumes of phosphate buffered saline (PBS) and then the solution was concentrated to recover the bacteria. DNA was isolated by methods with some modifications and the disclosed novel one-step multiplex PCR detection system was used. For the multiplex PCR analysis, a specimen was considered positive for H. pylori if 5 out of 10 DNA fragments or both DNA fragments from 2 out of the 5 loci were amplified. H. pylori positive results were achieved in 36% (4/11) from fresh raw chickens and 44% (8/18) from ready-to-eat raw tuna meat by using the multiplex PCR detection system. Results indicated that the test samples obtained from local grocery and restaurants were contaminated with H. pylori. In spite of the difficulty in culturing H. pylori by currently available methods, this is evidence that some raw foods and ready-to-eat foods may take an important role in the transmission route of H. pylori infection in humans.

### Example 4

### Urease-Pathology Negative (UPN) H. pylori Infection in Patients with Nonulcer Dyspepsia

Background & Aims: Many factors may account for the controversies with regard to the link between H. pylori infection and nonulcer dyspepsia (NUD). One important factor is the reliability of the diagnostic test for H. pylori. One-step multiplex PCR was developed and higher sensitivity and specificity has been achieved. This example explored effect of H. pylori treatment on NUD patients based on the indication given by the novel one-step multiplex PCR assay.

Patients and Methods:

41 patients with dyspepsia, who underwent EGD were diagnosed by CLOtest and regular pathological examination (immunohistochemistry and hemotoxin-eosin staining) as negative cases. However, diagnosed by one-step multiplex PCR, they were positive cases designated as UPN H. pylori patients and enrolled in the study. The group of patients was treated with Prevpac. About two to three weeks after treatment, these patients were examined with a second EGD, and the one-step multiplex PCR was performed on the biopsy. The severity of upper GI symptoms was measured on a ten-point scale. The treatment responses were evaluated in view of the proportion of patients who improved according to points on the initial dyspepsia summary score. Nonresponders, partial responders and total responders were designated according to less than 40%, between 40% and 80 % and more than 80% symptom improvement respectively.

Results: of 41 patients, 44% (18/41) had complete symptom responses, 39%(16/41) had partial responses, and 17% (7/41) had non-response. In 61% (25/41) patients, the second PCR detection on biopsy after treatment became negative.

Conclusions: These results showed that the H. pylori-positive patients who were revealed by one-step multiplex PCR in contrast to negative results from CLOtest and pathological examination do respond well to the treatment with Prevpac.

Treatment of UPN but PCR-postive patients

| Symptom response | N | PCR | n |
|---|---|---|---|
| Complete response | 18 (44%) | HP (−) | 13 |
| | | HP (+) | 5 |
| Partial response | 16 (39%) | HP (−) | 8 |
| | | HP (+) | 8 |
| Non-response | 7 (17%) | HP (−) | 4 |
| | | HP (+) | 3 |

### Example 5

### Extra-Ordinary Cases—Lives Saved, Job Restored and Problems Solved Due to Positive H. pylori Identified by Multiplex PCR

Background: Upper gastrointestinal diseases are well recognized and in majority of cases, etiologies are well recognized and treatments are successful. However, there are cases with no obvious etiologies, which make treatments difficult.

Aim: Multiplex PCR assay was helpful in diagnosis and treatments of seven extra-ordinary cases.

Method: A novel one-step multiplex PCR method was used to amplify 10 DNA fragments from five regions in the genome of H. pylori to diagnose H. pylori infection.

Case 1: A 58 years old white female, whose father had gastric carcinoma, had epigastric pain for 2 years. She had been on PPI but there was no improvement. Biopsies were performed for multiplex PCR which were positive, but negative based on CLOtest and pathology. She was treated with PrevPac for 2 weeks with total resolution of symptoms, and repeat gastroscopy showed negative PCR for H. pylori.

Case 2: A 31 year old white female who had epigastric pain for many years. Multiple gastroscopies revealed gastritis and esophagitis. PPI did not improve her symptoms. Gastoscopy showed H. pylori by multiplex PCR. Two weeks of PrevPac totally eradicated the old symptoms and H. pylori.

Case 3: A 55 year old policeman who suffered from GERD with regurgitation for 6 months and the pain of suffocation. He had gastroscopy which showed positive H. pylori by multiplex PCR. Two weeks of PrevPac eradicated the H. pylori and 80% symptom resolution.

Case 4: A 53 year old white male salesman had a two month history of GERD with hoarseness. He had to quit his job because of the pain. Gastroscopy showed H. pylori by multiplex PCR. He was treated with PrevPac for 2 weeks with some improvement. He was then treated with PPI, tetracyclemyin, metronidagole, peptobismol with 80% improvement. He returned back to work.

Case 5: UB is a 70 years white male biochemist who has suffered a lump in the throat and was diagnosed by ENT to have GERD. He was treated with different PPI's with no improvement of symptoms. Gastroscopy showed H. pylori by multiplex PCR. Treatment with PrevPac gave 80% of the resolution of symptoms but still a positive H. pylori.

Case 6: FK is a 70 year old white man who had been admitted to the hospital every two weeks for recurrent bleeding from gastritis and gastric ulcer in spite of being on patoprazole. Gastroscopy showed H. pylori only by multiplex PCR. 2 weeks of PrevPac treatment eradicated H. pylori and recurrent upper GI bleeding also resolved.

Case 7: MS is a 42 year old singer, who had symptoms of GERD with loss of her voice for two years. She did not respond to PPI, and gastroscopy showed H. pylori by multiplex PCR, and two weeks of PrevPac partly improved her symptoms and two more weeks of tetracyclemyin/metronidagole/prevacid/peptobismol improved her symptoms so that she resumed her singing career.

Conclusion: Diagnosis of H. pylori can be difficult. The negative CLOtest and pathology may not rule out H. pylori infection. The above cases demonstrate that multiplex PCR is the ultimate diagnostic test to clarify the presence of H. pylori infection. Accurate diagnosis provides adequate treatments, which resist in extraordinary treatment cases.

### Example 6

### Detection of H. pylori in Urease Test Negative Gastric Samples by Novel Multiplex PCR Assay

Background and Aims: All patients with dyspepsia, either newly or previously diagnosed, should be tested for H. pylori infection. The CLOtest assay is widely used in clinical practice to detect the urease enzyme of H. pylori in gastric mucosal biopsies and many physicians consider it as a gold standard method. However, this is not sensitive enough to diagnose H. pylori infection. The one-step multiplex PCR detection system described herein can amplify 10 DNA fragments from 5 DNA regions in the genome of H. pylori at the same time. The diagnostic value of this new multiplex PCR assay to detect H. pylori infection was assessed, and the negative results from the CLOtest were evaluated.

Methods: Urease test-negative gastric specimens, which had been assayed by CLOtest from 276 individuals with dyspepsia symptoms undergoing endoscopy. The CLOtest was performed first and if the result was negative, the negative specimen was collected from the CLOtest gel. From this specimen, the DNA was isolated and the one-step multiplex PCR was performed.

Results: Positive results were achieved in 42% (116/276) with multiplex PCR from the H. pylori negative samples tested by CLOtest.

Conclusions: Multiplex PCR method is a highly specific and sensitive method in the detection of H. pylori. The results of H. pylori tested by current methods should be carefully reviewed to ensure that the result is not a false-negative to obtain accurate diagnosis the negative samples of CLOtest should be further confirmed by the multiplex PCR assay.

### Example 7

### Detection of H. pylori at Distal Esphageal Mucosa by a Novel Multiplex Pcr Assay

Background and Aims: Many factors have been reported to affect the ability to identify H. pylori in clinical practice. These include the type of tests used, the experience of the pathologist, the number of biopsy samples, and the location from which they were taken. H. pylori is a highly motile and most commonly lives in the mucous layer of the gastric epithelium. Motility, by way of flagella, enables the organism to easily travel within the mucous layer. H. pylori in the esophagus is found only in areas of Barrett's esophagus. In order to determine the prevalence of Helicobacter pylori infection at antral gastric and esophageal mucosa and its correlation with the multiplex PCR findings, a prospective study was performed with a novel one-step multiplex PCR method described herein in 102 patients with dyspepsia symptoms undergoing endoscopy.

Methods:. Six specimens, 2 from the corpus, 2 from the antrum and 2 from the distal esophageal, were collected from each patient by endoscopy for extracted DNA and was performed the one-step multiplex PCR.

Results: Helicobacter pylori was found on the biopsy specimens of the gastric antrum/corpus and distal esophagus in 43% (44/102) and 32% (33/102), respectively. The combined positive results of the three locations was 53%.

Conclusions: H. pylori could live in the distal esophagus since some cases have positive H. pylori in the esophagus but not in the stomach. (Although false positive results may be obtained, the samples of distal esophagus due to contamination from the gastric antrum/corpus in the process of endoscopy or because of the hypersensitivity of the PCR method.) Therefore multiple biopsies, especially the specimens from distal esophagus may be needed for the diagnosis of H. pylori infection in some situations.

### Example 8

### Diagnosis of H. pylori Infection in UPN (Urease-Pathology Negative) Patients by Novel Multiplex PCR Assay

Background: The diagnosis and treatment of NUD (Non-ulcer Dyspepsia) are very controversial because of the unknown etiology. In addition, the treatment with PPI is not very effective. A prospective study was performed with a novel one-step multiplex PCR method, which can amplify 10 DNA fragments from 5 DNA regions in the genome of H. pylori at the same time, in UPN (CLOtest and standard immunohistochemistry negative) patients with dyspepsia symptoms to elucidate the prevalence of UPN H. pylori infection.

Methods: This study was performed in 378 patients with dyspepsia symptoms undergoing endoscopy. Group 1 includes 102 patients, each with six fresh specimens, 2 from the corpus, 2 from the antrum and 2 from the distal esophageal. Group 2 includes 107 patients' specimens collected from the negative CLOtest gel, 2 from the antrum and 1 from the distal esophageal. Group 3 includes 169 patients' specimens collected from the negative CLOtest gel with two biopsies from the antrum. In the second and third groups, the CLOtest was performed first, and if the CLOtest was negative (waiting for more than 24 hours), the specimen was collected from the CLOtest gel and then the one-step multiplex PCR was performed.

Results: Helicobacter pylori was found, in the fresh biopsy specimens, 43% (44/102) in the gastric antrum/corpus and 32% (33/102) in the distal esophagus, and the combined positive results of the three locations achieved was 53%. Positive results were 46% (49/107) in group 2 and 40% (67/169) in group 3. The total positive results of the group 2 and 3 were achieved in 42% (116/276) with the multiplex PCR assay described herein.

Conclusions: The multiplex PCR method described herein is a highly sensitive method in the detection of UPN H. pylori infection. Regardless of the methods of specimen collection, in the setting of dyspepsia, 42-53% of the CLOtest and pathology-negative patients are positive for H. pylori by PCR. It would be better if the negative samples of CLOtest were further confirmed by the multiplex PCR assay to ensure the correct diagnosis. There was no statistically significant different between group 1 and group 2 plus 3, however, if the PCR from the CLOtest sample is negative and H. pylori is still suspected, fresh sample for H. pylori may still be able to give a positive H. pylori. Since fresh samples seem to have a higher yield in positive H. pylori (Although not statistically significant) detection.

### Example 9

### Detection of Coccoid Form of H. pylori in UPN-HPI Patients

The main objective of this example is to establish a fast, sensitive, and specific method for detecting Helicobacter pylori infection and directing the treatment for clinical patients.

Since its discovery in 1982 by Marshall and Warren, H. pylori have been recognized to play a significant pathogenic role in active chronic gastritis, the development and recurrence of gastric and duodenal ulcers, and perhaps gastric cancer and cardiovascular disease. The prevalence of H. pylori infection is worldwide and maybe as high as 80% in developing countries and up to 40% in developed countries.

Currently, H. pylori can be detected by different tests, including bacterial culture, histology, serology, stool antigen test, rapid urease test, isotope urea breath test, and PCR method. However, all the methods have their advantages and disadvantages regarding sensitivity, specificity, convenience, cost, and immediacy. Of these tests, the PCR method is advantageous in most aspects of pathogen detection. It is highly sensitive and specific comparing to the urease assays, which is not sensitive and not specific in the presence of other urease-positive bacteria. It is cheap if performed in bulk, and it could produce results much faster than bacterial culture, as culturing H. pylori is lengthy and not possible in most situations. In addition, PCR test for various bacteria, including H. pylori, are available for a broad spectrum of specimens, including laboratory cultures and clinical samples.

However, potential problems with the traditional PCR include false positives due to contamination, homologous DNA sequences among various species, unspecific amplifications, and false negatives due to reaction failures. According to a recent study, all 20 of the biopsied samples which were studied were UreC positive by traditional PCR, but only 70% were urea positive and only 45% were hpaA positive by PCR. Excluding the possibilities of the presence of PCR inhibitors or errors in PCR manipulation, polymorphism at the primer's binding site could be one plausible explanation for such different results.

To prevent the potential problems of conventional PCR and improve the diagnosis of H. pylori, a more advanced system for the diagnosis of H. pylori, described herein known as the multiplex PCR system. The basic idea of the PCR system is to amplify selected gene loci in more than one region at the same time. By this method, PCR sensitivity is increased, as the chances of amplifying the selected DNA regions are much higher than the chances of amplifying only one region. False negatives and false positives are revealed in multiplex amplification because each amplicon provides an internal control for the other amplified fragments. Design of additional primers for the system increases its specificity. An amplicon of a sequence conserved among several pathogens is often included in the single locus PCR, and it could give a false positive. Multiplex assays with different locus combinations could distinguish a particular pathogen from others. Moreover, confirming the amplified DNA fragments by nested PCR at the same time will lend to more confidence in diagnosing patients with H. pylori infection.

So far two forms of Helicobacter pylori have been described: the spiral multiflagellate and the coccoid dormant form. Nutrient deprivation, exposure to anti-ulcer drugs and antibiotics, extended incubation, pH adjustments, and attachment to the gastric epithelium have been found to be significant ways in which H. pylori is able to convert and morph from spiral-shaped H. pylori to coccoid forms. A number of characteristics of the coccoid form appear relevant to its biological niche and clinical correlations. First, there is a loss of mRNA for urease, suggesting an adaptation to low-acid environment. Second, the coccoid binds less well to epithelial cells and initiates reduced mucosal inflammation and lower levels of antibody. Third, it is less susceptible to eradication by antibiotics, suggesting a link to failed eradication therapy. Fourth, some, but not all, therapies used to manage dyspepsia appear to favour induction of the coccoid state. These include proton pump inhibitors, bismuth salts and penicillins, but not the imidozales and macrolides. The link with failure to respond to eradication therapy needs clarification. Fifth, serological assays to detect “classical” H. pylori infection (using gold standard criteria) in older subjects are known for their high sensitivity, but low specificity caused by “false”-positive antibody assays. Finally, altered surface proteins may radically affect outcome of the host-parasite relationship. It appears that coccoids induce a lower level of immune response, but qualitative aspects of host effector mechanisms remain unknown.

Since the coccoid form H. pylori may play an important role in transmission of the infection and may account for some treatment failure and relapses, it is important to diagnose the coccoid form H. pylori. Due to the characteristics of coccoid form of H. pylori, the tests based on culture, urease assay and antigen detection may generate false-positive or false-negative results. The novel multiplex PCR detecting system is a test based on DNA. It can also be used to the detect coccoid form H. pylori.

Subjects and Specimens

Five-hundred randomly selected subjects, over the age of 18, with no discrimination regarding gender and racial/ethnic composition are chosen for this study if they are admitted to the Gastointestinal Laboratory (G.I. Lab) to undergo elective Esphogastroduodenoscopy (EGD) because they present with symptoms, such as abdominal pain, burning feeling in the stomach, nausea and vomiting, abdominal bloating, belching, and ulcer-related indigestion, which are commonly associated with H. pylori infection and symptoms of gastroesophageal reflux disease (GERD). The Consent is obtained from each subject by the principal investigator or a member of the research staff only after the consent form is fully explained and any concerns and/or questions are answered. The subject is sedated and undergoes EGD examination. 4 biopsies from the antrum and 2 from the body are taken for the study.

Multiplex PCR System Primer Design

An important aspect of the PCR protocol lies in the choice of primers. After reviewing several published primers that are specific for H. pylori genes and searching the GenBank, the following loci were based as amplification targets: the urease gene (urea), 26 kDa protein antigen, Hpa Agene, 0.86kb DNA fragment, and DNA sequences of 16S ribosomal RNA.

For each locus, one forward primer is selected as the common primer (FC) and two reverse primers (R1,R2), wherein R2 is inside of the amplifying region of R1 and FC (FIG. 10).

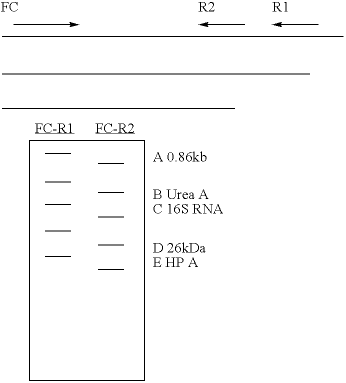

A, B, C, D, and E are five selected DNA loci of H. pylori. Each locus has two bands but in different lanes. The lower band is the same as nested PCR product of the larger band in each locus.

All 5 R1 primers and 5 R2 primers are mixed with 5 FC primers respectively, well balanced, and set in two separate amplification systems. This allows 5 DNA fragments to be amplified in each tube at the same time.

The Taq DNA Polymerase that is used may be either a heat-activated enzyme like Taq Gold Polymerase or Immolase DNA Polymerase because the hot-start PCR method is used with multiplex PCR. The touch-down PCR method is also used.

Based on the proposal made by Monteiro et al, in which a traditional PCR diagnosis is considered positive when one of two biopsied specimens from each part of the stomach is positive using two sets of primers derived from two genes, the one-step multiple-nested PCR assay is positive for H. pylori if 5/10 fragments (at least cover three genes).

Detection of Coccoid form of H. pylori

The CloTest (based on urease) and regular immunostaining can not detect the coccoid form of H. pylori. Two methods are used to locate those bacteria in the biopsy tissue. The formalin-fixed and paraffin-embedded sections is prepared and used as in the regular immunostaining. If there are difficulties, the fresh tissue and frozen sections are used. All the specimens in urease-negative and pathology-negative (regular examination) patients will be examined.

Indirect Immunofluoresence

The paraffin sections are depareffinised and rehydrated. After air drying, 50 μl of 0.01% (w/v) pronase in PBS is added onto the section to expose antigen, and incubated at room temperature for 2 min. 50 μl of diluted antibody (murine monoclonal antibody against H. pylori) is added and incubated for 60 min. The slides are then washed by dipping the slides in PBS six times. 50 μl of goat anti-mouse IgG conjugate (1:100 dilution) as secondary antibody are added and incubated for another 60 min. After washing as above, the slides are air-dried and I drop of SlowFade is added, then are examined with a fluorescent microscopy.

Immunogold Silver Enhancing Stain

The depareffinised and rehydrated sections are treated with 5% aminopropyl and then are incubated in ethanol for 3 min. The sections are immersed in the mixture of 0.5% normal goat serum and 0.05% bovine serum albumin in PBS. The sections are then reacted with mouse IgG fraction specific for H. pylori for 2 hours. After washing in PBS, sections are rinsed in 5 nm gold particle labeled goat antiserum specific for mouse IgG. After washing, the sections are enhanced by silver enhancing kit (British Biocell) for 8 min. Then the slides are conterstained with hematoxlin.

Medical Chart Review

Medical records are obtained and reviewed for each subject that provides gastric biopsies. The purpose of this medical record review is to obtain information pertaining to the subject's symptoms upon admittance, diagnosis before and after EGD, and test results.

Data Collection and Statistical Analysis

The sensitivity and specificity relative to CloTest and regular pathology examination are calculated. The significance of the difference of rates between two groups is analyzed by the Fisher's exact test.

### Example 10

### Detection of Helicobacter pylori by Multiplex PCR: Comparison with Immunohistochemistry and CLOtest

Helicobacter pylori is associated with up to 95% of duodenal and gastric ulcers and with the development of gastric carcinoma and MALT lymphoma. There are a wide variety of tests available for detecting this important organism. The aim of this study is to compare the detection ability of a novel PCR assay with standard methods of immunohistochemistry (IHC) and CLOtest.

Upper endoscopy was performed on 61 patients (30M,31F ages 18-94) for symptoms of dyspepsia. Biopsies of the antrum/body and/or gastroesophageal (GE) junction were submitted for routine histology. The degree of mononuclear and PMN inflammation was scored [none (1), mild (2), moderate (3), severe (4)] and the presence of Helicobacter pylori was evaluated by immunohistochemistry (Novocastra). Separate paired biopsies of the antrum/body (n=57) and GE junction (n=45) were evaluated by a novel one-step multiplex PCR assay examining 5 loci in the H. pylori genome. Separate antrum biopsies were submitted for CLOtest and interpreted using standard methodology.

Sample Section

61 patients (30M, 31F; range 18-94 years) underwent esophagogastroduodenoscopy for symptoms of dyspepsia. Biopsy samples were taken from the antrum/body and/or gastroesophageal junction

CLOtest

CLOtest was performed on separate biopsies samples taken from the antrum/body and interpreted using standard methodology

Histologic Examination

Biopsies were submitted for routine histologic examination with H&E stain. The degree of mononuclear and polymorphonuclear activity was scored using the Updated Sydney System: Classification and Grading of Gastritis (MF Dixon et al. Am j Surg Pathol 20(10): 1161-1181, 1996).

Immunohistochemistry

Charged unstained slides were evaluated by immunohistochemistry using standard techniques with a rabbit polyclonal antibody against heat treated cells of Helicobacter pylori strain CH-20 429 (Novocastra).

Separate paired biopsy samples were analyzed using a novel multiplex PCR method In this system, five loci in the Helicobacter pylori's genome were chosen as amplification targets. The Hpa A gene, 26 kDa protein antigen, 16S ribosomal RNA, urease A gene and a specific 0.86kb DNA fragment were the loci chosen as amplification targets. For each locus, one forward primer (FC) and two reverse primers. (R1 and R2) were designed and two fragments, one internal to another are expected to be amplified (FIG. 10).

59% (36/61) of patients overall were PCR(+) in antrum/body and/or GE junction. 59% (36/61) of patients overall were diagnosed with Helicobacter pylori by PCR in antrum/body or GE junction biopsiesComparison with IHC and CLOtest is provided in TABLE 3. All GE junction IHC(+) PCR(−) were antrum/body PCR(+). TABLE 4 lists the inflammation score in the various subgroups.

PCR accurately identifies Helicobacter pylori detected by IHC and CLOtest. PCR detects the presence of H. pylori in a significant number of cases in which the organism is not identified by these routine methods. IHC(−) CLOtest(−) PCR (+) biopsies show inflammation scores similar to IHC(−) CLOtest(−) PCR (−). In the antrum/body most IHC(−) CLOtest(−) PCR(+) are histologically normal. Inflammatory cell activity is not a useful predictor of infection for immunohistochemistry negative biopsies.

### Materials and Methods

For each locus, one forward primer was selected as the common primer (FC) and two reversed primers (R1 and R2) were selected in which R2 is inside the amplifying region of R1. In addition, these primers met the following criteria: 1) Tm, around 60° C., 2) G+C, about 50%,3) length, 20-30 nt, 4) no dimer formations with other primers, 5) few mutations in the primer binding site after checking the published H. pylori DNA sequences, and 6) specific for H. pylori.

To better balance the primer mixture, the primers were divided into two groups: group A, with all the five FC primers and five R1 primers and group B, all the five FC primers with five R2 primers. Under these settings, 10 DNA fragments were amplified in two tubes at the same time.

Helicobacter pylori and other bacterial strains used. Three H. pylori stains ordered from American Type Culture Collection (Nanassas, Va., USA): ATCC 43504, ATCC 700392 (26695) and ATCC 700824 (J99), and eleven common bacterial species, 1) Escherichia coli, 2) Enterobacter cerogenes, 3) Enterobacter cloacae, 4) Enterococcus spp., 5) Virdians Group, Stereptococcus, 6) Pseudomonas aeroginosa, 7) Serratia spp, 8) Klebsiella pneumoniae, 9) Methicillin resistant Staphylococcus aureus, 10) Lactobacillus spp. and II) Citrobacter spp., which were isolated from clinical samples at Evanston Northwestern Healthcare, Evanston Hospital (Evanston, Ill.), were selected as templates to assay the accuracy, sensitivity, specificity, and reproducibility of the one-step multiple-nested PCR.

DNA Extraction. The DNA of H. pylori and other bacteria were isolated according to the published method with some modifications disclosed herein.

Buffers and Solutions: Ethanol, Potassium acetate (5 M), TE (pH 7.6), Cell lysis buffer: 10 mM Tris-Cl (pH 8.0), 0.05M EDTA (pH 8.0), 0.5%(w/v) SDS, 20 μg/ml DNase-free RNase. Other enzymes and buffers include DNase-free RNase (4 mg/ml), Proteinase K (20 mg/ml), DNA from mammalian tissue such as human gastric biopsy specimens containing Helicobacter pylori and other bacteria culture were extracted following the method disclosed herein.

The gastric tissue was ground with a small glass tissue grinding bar in a 1.5 ml eppendorf tube. For cultured Helicobacter pylori or other bacteria, one or two colonies were picked and introduced into a 1.5 ml tube with 50 μl TE. The tubes were heated at 100° C. for 5 minutes. Then 500 μl cell lysis buffer was added to the tubes followed by an addition of 3 μl of proteinase K solution to the lysate. The digest was incubated at 56° C. for 1 hour or for no longer than 16 hours at room temperature. The sample was allowed to cool to room temperature. Then, 200 μl of potassium acetate solution was added to the sample and the contents were mixed by vortexing the tube vigorously for 20 seconds.

The precipitated protein/SDS complex was pelleted by centrifugation at maximum speed (approximated 10,000 g) for 3 minutes at 4° C. in a microfuge. A pellet of protein was visible at the bottom of the microfuge tube after centrifugation. If no pellet was seen, the lysate was incubated for 5 minutes on ice and the centrifugation step was repeated. The clear supernatant was transferred to a fresh microfuge tube containing 500 μl of chloroform. The solution was mixed well and then centrifuged at maximum speed for 5 minutes at room temperature in a microfuge.

The upper aqueous phase was transferred to a fresh centrifuge tube and 900 μl of absolute ethanol was added to the tube. The tube was inverted several times and centrifuged at maximum speed for about 5 minutes at room temperature in a microfuge. The supernatant was removed by aspiration and 500 μl of 70% ethanol was added to the DNA pellet. The tube was inverted several times and centrifuged at maximum speed for 1 minute at room temperature in a microfuge.

The supernatant was removed without disturbing the pellet by aspiration and the DNA pellet was allowed to dry in air for about 15 minutes or until dry. The DNA pellet was redissolved in 50-100 μl of TE (pH 7.6).

Touch down PCR: The following PCR program was used for a touch down PCR: Initial denaturizing at 94° C. for 5 minutes, followed by the first 6 cycles at 94° C. for 40 seconds, 66° C. for 30 seconds and the annealing temperature is reduced from 66° C. to 61° C. by 1° C. per cycle, 72° C. for 50 seconds. Then approximately 36-40 cycles were repeated as follows: 94° C. 40 seconds, 56° C. 30 seconds, 72° C. 50 seconds, 72° C. 10 minutes, 4° C. hold. The PCR thermo cycle device used was of model Eppendorf Mastercycler 5333 (Eppendorf Scientific, Inc., Westbury, N.Y.).

Software used for primer design: DNASTAR, Lasergene® sequence analysis software, PrimerSelect 4.05 (DNASTAR, Inc., Madison, Wisc. 53715).

Balancing the concentration of primer mix: Each primer solution was prepared at a concentration of 10 pmol/μl. Equal volume of the two primers for each locus was added to the PCR mix. The amplification of individual locus was tested and according to the results the concentration of primers was adjusted for subsequent experiments. Primers corresponding to multiple loci were mixed and the multiplex PCR was performed. Depending on the results, the final primer concentration was adjusted. For each primer, the concentration was about 0.05-0.1 pmol/μl for the multiplex PCR. The final concentrations of primers were tested under different PCR conditions to make sure that the primer mix would work well under a range of conditions, such as the amount of DNA template, polymerase, Mg⁺⁺ concentration, and a range of annealing temperatures and the like.

Immunomagnetic separation of H. pylori from human feces and DNA extraction: An exemplary immuno-separation and H. pylori DNA extraction is described in Monteiro et al., (2001), the disclosure is herein incorporated by reference. Stool specimens were suspended at 1.5:5 (wt/vol) for solid and semisolid samples and 1.5:5 (vol/vol) for liquid in phosphate-buffered saline and incubated overnight, under agitation at room temperature. The suspension was then filtered through three layers of cotton gauze and used for immunomagnetic separation of H. pylori. Briefly, magnetic uncoated beads were coated with rabbit anti-H. pylori immunoglobulin at a concentration of 5 μg of antibody for 10⁷ Dynabeads according to the procedure recommended by Dynal. A 60-μl volume of coated Dynabeads was mixed with 1 ml of fecal suspension and incubated at 4° C. with continuous shaking for 2 h. The coated Dynabeads were recovered by magnetic force with a Dynalmagnet and then suspended in the lysis buffer of the QIAamp tissue kit for DNA extraction. Twenty microliters of a proteinase solution (20 mg/ml) was then added, followed by incubation at 56° C. for 2 h. A second buffer provided in the kit was added, and the sample was incubated at 70° C. for 10 min. Next, 200 μl of ethanol was added, and the suspension was loaded on the QIAamp spin column followed by a centrifugation at 6,000× g for 1 min. The QIAamp spin column was placed in a 2-ml collection microtube, and the tube containing the filtrate was discarded. The column material was washed twice (250 μl each) with the first washing buffer and twice (250 μl each) with the second washing buffer provided in the kit. Finally, the DNA was eluted with 100 μl of distilled water preheated to 70° C. (2×50 μl).

Amplification (Polymerase Chain Reaction, PCR): The template DNA was added to 20 μl of a reaction mixture containing 10 mM Tris-HCl (pH 8.3), 50 mM KCl, 1.6 mM MgCl₂, 0.001% (w/v) gelatin, 0.3 mM each deoxynucleotide, 0.05 μM each oligonucleotide primer. Immolase™ DNA polymerase (5.0 U; Bioline Ltd, London, UK) was used. The PCR was performed with a PCR thermal cycler (Marstercycler, Eppendorf Scientific Inc, Waterbury, N.Y., USA) and the PCR conditions were as follows: an initial denaturizing at 94° C. for 5 minutes, followed by the first 6 cycles at 94° C. for 40 seconds, 66° C. for 30 seconds and the annealing temperature is reduced from 66° C. to 61° C. by 1° C. per cycle, 72° C. for 50 seconds. The final extension step was at 72° C. for 10 min.

For example, two master mixes that each contain 4.0 μl of 5× PCR buffer, 1.0 μl of primer mix M1 or M2, 0.6 μl of 10 mM dNTPs and 1.0 μl of DNA polymerase (5U) were prepared. M1 contained for example F1/R1, F2/R2, F3/R3, F4/R4 and F5/R5, and M2 contained F1/RN1, F2/RN2, F3/RN3, F4/RN4 AND F5/RN5 (TABLE 2). Depending on the number of samples tested, the master mix was scaled-up. 1.0 ml of sample DNA isolated from the specimens were added to the master mix. The PCR was performed as described above.

Direct identification of cultured H. pylori: For direct detection of H. pylori from culture, a colony was selected with a pipette tip and was mixed in 100 μl Tryptic Soy Broth or TE solution in a 0.5 ml test tube. Then 1 μl of the cell solution was directly added to a PCR tube with 19 μl PCR reaction mixture. Amplification was performed as disclosed herein.

Analysis of the multiplex PCR products: The analysis of PCR products was carried out by agarose gel electrophoresis. the PCR products were analyzed on a 2.0% agarose gel by electrophoresis of a 10 μl aliquot and stained with ethidium bromide and visualized by excitation under UV light. To conveniently compare the size of the amplified PCR products, previously amplified and confirmed PCR products from known H. pylori DNA were as used in addition to a commercial 100-bp DNA ladder.

Assaying the sensitivity of the one-step multiple-nested PCR: The sensitivity of the one-step multiple-nested PCR was investigated with serial dilutions of the H. pylori DNA, followed by amplification with the one-step multiple-nested PCR in the conditions disclosed herein.

Assaying the specificity of the one-step multiple-nested PCR: To test the specificity of the multiplex PCR assay, 11 bacterial species were chosen, which included: 1) Escherichia coli, 2) Enterobacter aerogenes, 3) Enterobacter cloacae, 4) Enterococcus AP, 5) Virdians Group, Stereptococcus, 6) Pseudomonas aeruginosa, 7) Serratia species, 8) Klebsiella pneumoniae, 9) methicillin resistant Staphylococcus aureus, 10) Lactobacillus spp. and 11) Citrobacter spp. as templates. The PCR was performed in the conditions described for system 2 (two tubes with five FC primers and five R primers each) along with H. pylori J99 as a positive control.

Comparing the CLOtest and histology method: Ninety stomach biopsy samples were collected from the patients with dyspepsia symptoms undergoing endoscopy (informed consent was obtained). To overcome the problem of patchy H. pylori distribution (so that the organism is not captured in the tested tissue sample), the same gastric specimen was used for both CLOtest and PCR assay. Standard CLOtest methods were used. The CLOtest was performed first and after obtaining the results (waiting from about 20 minutes to 24 hours), specimens were collected from the CLOtest plates and DNA was isolated using standard DNA extraction procedures and the one-step multiplex PCR was performed. Other specimens were sent to the clinical laboratory for the histology study performed by an expert clinical pathologist.

Multiplex PCR Design. A goal of the disclosed PCR method is to overcome false-negative results by amplifying more than one region at the same time because the possibility to amplify all or some of the selected DNA region is much higher when multiple regions are used rather than only one region. Moreover, the amplified DNA fragments can be confirmed using the internal control to rule out false-positives.

Primer Design. To design the multiplex PCR primers, several published primers were reviewed that are specific for H. pylori genes and the GenBank was searched. The urease genes (urea), 26 kDa protein antigen, Hpa A gene, 0.86 kb DNA fragment and DNA sequences of 16S ribosomal RNA were chosen as amplification targets.

TABLE 1
Sensitivity and Specificity of CLOtest and
Histology relative to Multiplex PCR method.

| | | Multiplex PCR | | | |
|---|---|---|---|---|---|
| | N | (+) | (−) | Sensitivity | Specificity |
| CLOtest | | | | | |
| (+) | 39 | 38 | 1 | 38/61 = 0.62 | 28/29 = 0.97 |
| (−) | 51 | 23 | 28 | | |
| Total | 90 | 61 | 29 | | |
| Histology | | | | | |
| (+) | 10 | 10 | 0 | 10/61 = 0.16 | 29/29 = 1.0 |
| (−) | 80 | 51 | 29 | | |
| Total | 90 | 61 | 29 | | |

TABLE 2
Characteristics of primers and amplicons from five loci in H. pylori

| | | | | | |
|---|---|---|---|---|---|
| | | | Length | Amplicon | H. pylori 26695 |
| Locus | Primer | 5′-3′ Sequence^{c} | Tₘ °C. | Size^{a} | (position)^{b} |

| | | | | | | | |
|---|---|---|---|---|---|---|---|
| 0.86 kb | F1 | AACGC CGTGA GTTCG TCGTA TCG | 23 | 61.9 | | 80650-80628 | |
| (ureC | R1 | CATAT AGCCG CTTTT TCTGG TGTCT TTA | 28 | 59.2 | 706 bp | 79945-79972 | |
| and prfA) | RN1 | CCTCA CGCCA TCAGT CCCAA AAAT | 24 | 61.9 | 574 bp | 80077-80100 | |
| Ure A | F2 | TGATA GGCAA GCAGA CAACG AA | 22 | 56.3 | | 77351-77330 | |
| gene | R2 | GATGT GTGTG TCAAT ACCAC CAGC | 24 | 62 | 526 bp | 76826-76849 | |
| | | RN2 | GCACG ACCCA CGCTA AGATT GT | 22 | 57 | 465 bp | 76887-76908 |
| 16S | F3 | CAGGT CGCCT TCGCA ATGAG TA | 22 | 57.2 | | 1511958-1511937 | |
| rRNA | R3 | ACGGG AGGCA GCAGT AGGGA ATA | 23 | 59.8 | 371 bp | 1512284-1512306 | |
| | RN3 | GCCGC GTGGA GGATG AAGGT | 20 | 60.0 | 315 bp | 1512232-1512251 | |
| 26 kDA | F4 | TCATG CCTTT ATCGC CTTTT CTCC | 24 | 59.5 | | 1645764-1645741 | |
| | R4 | GTGGA AAAAG GCGGT ATCGG TCAA | 24 | 61.9 | 277 bp | 1645488-1645511 | |
| | RN4 | CGATC GCTTT GAGAG GTGCT TTTT | 24 | 59.8 | 183 bp | 1645582-1645603 | |
| Hpa A | F5 | CTAGA GCCTA TGAGT GGGGA ATCTT T | 26 | 58.0 | | 854235-854210 | |
| | R5 | ATCCG TTCCC TTAAC CATAG TGCT | 24 | 58.0 | 138 bp | 854098-854121 | |
| | RN5 | TGCTA ACTAA CCCCC CGCTA TGGC | 24 | 57.2 | 118 bp | 854118-854141 | |

^{a}-The “F” primers serve common to the “R” and “RN” primers. “RN” primers refer to the nested internal primers.
^{b}-position refers to the beginning and end of primer sequences in H. pylori 26695 genome (Genbank accession number AE000511.1).
^{c}-bold letter denotes mutations in the published sequence of H. pylori.

TABLE 3
Summary of Results of Biopsy Samples from All 61 Patients
by PCR, CLOtest, and Immunohistochemistry (IHC)

| | Antrum/body | Antrum/body | GE junction | GE junction |
|---|---|---|---|---|
| H. pylori | PCR (+) | PCR (−) | PCR (+) | PCR (−) |
| IHC (+) | 11 | 0 | 1 | 4 |
| IHC (−) | 18 | 28 | 14 | 26 |
| CLOtest (+) | 13 | 0 | Not done | Not done |
| CLOtest (−) | 19 | 27 | Not done | Not done |

All patients with GE junction biopsies that were PCR (−) and IHC (+) were PCR (+) with their paired antrum/body biopsy. All IHC (+) and/or CLOtest (+) antrum/body biopsies were PCR (+) 56% (20/36) of patients diagnosed with H. pylori by PCR were negative by standard histologic evaluation and CLOtest (TABLE 3).

TABLE 4
Summary of Inflammation Score in the Various Subgroups

| | Inflammation Score | | | | |
|---|---|---|---|---|---|
| | Antrum/Body | | | GE junction | |
| | PCR(+) | PCR(−) | PCR(+) | PCR(+) | PCR(−) |
| | IHC(+) | IHC(−) | IHC(−) | IHC(+) | IHC(−) |
| PMN | 36% | 100% | 94% | 100% | 96% |
| cells ≦ 2 | (4/11) | (28/28) | (17/28) | (1/1) | (25/26) |
| PMN | 64% | 0% | 6% | 0% | 4% |
| cells ≧ 3 | (7/11) | (0/28) | (1/18) | (0/1) | (1/26) |
| Mononuclear | 0% | 82% | 83% | 0% | 31% |
| cells ≦ 2 | (0/11) | (23/28) | (15/18) | (0/1) | (8/26) |
| Mononuclear | 100% | 18% | 17% | 100% | 69% |
| cells ≧ 3 | (11/11) | (5/28) | (3/18) | (1/1) | (18/26) |

A majority of IHC (+) samples had inflammation scores >2 for both mononuclear and polymorphonuclear activity irrespective of biopsy site and PCR results IHC (−) biopsies that were PCR (+) demonstrated a similar pattern of mononuclear and polymorphonuclear activity to PCR (−), IHC (−) biopsies at the antrum/body and GE junction (TABLE 4).

### DOCUMENTS CITED

The following documents are incorporated by reference to the extent they relate to protocols used in this disclosure.
Andersen L P, Dorland A, Karacan H, Colding H, Nilsson H O, Wadstrom T, Blom J. Possible clinical importance of the transformation of Helicobacter pylori into coccoid forms. Scand J.Gastroenterol. 2000;35(9):897-903.
Clayton C L, Kleanthous H, Coates P J, Morgan D D, Tabaqchali S. Sensitive detection of Helicobacter pylori by using polymerase chain reaction. J Clin Microbiol 1992;30(1):192-200
Cover T L, Blaser M J. Helicobacter pylori: a bacterial cause of gastritis, peptic ulcer disease, and gastric cancer. ASM News 1995;61:21-6
Dubois A. Spiral bacteria in the human stomach: the gastric Helicobacters. Emerg Infect Dis 1995;1:79-84
Dunn B E, Cohen H, Blaser M J. Helicobacter pylori. Clin Microbiol Rev 1997; 10(4):720-741
He Q, Wang J P, Osato M, Lachman L B. Real-time quantitative PCR for detection of Helicobacter pylori. J Clin Microbiol. 2002;40(10):3720-8.
Ho S A, Hoyle J A, Lewis F A, Secker A D, Cross D, Mapstone N P, Dixon M F, Wyatt J L, Tompkins D S, Taylor G R, Quirke P. Direct polymerase chain reaction test for detection of Helicobacter pylori in humans and animals. J Clin Microbiol 1991 ;29:2543-2549
Hua J S, Bow H, Zheng P Y, Yeoh G K, Ng C H, Lim G S. Coexistence of Helicobacter pylori spiral and coccoid forms in experimental mice. World J Gastroenterol. 1998;4(6):485-488.
Li C, Musich P R, Ha T, Ferguson D A Jr, Patel N R, Chi D S, Thomas E. High prevalence of Helicobacter pylori in saliva demonstrated by a novel PCR assay. J Clin Pathol 1995;48:662-6
Marshall B J, Warren J R. Unidentified curved bacilli in the stomach of patients with gastritis and peptic ulceration. Lancet 1984; 1:1311-5
Monteiro et al., (2001) Magnetic immuno-PCR assay with inhibitor removal for direct detection of Helicobacter pylori in human feces. J Clin. Microbiol.; 39(10):3778-80.
Myung S J, Kim M H, Shim K N, Kim Y S, Kim E O, Kim H J, Park E T, Yoo K S, Lim B C, Seo D W, Lee S K, Min Y I, Kim J Y. Detection of Helicobacter pylori DNA in human biliary tree and its association with hepatolithiasis. Dig Dis Sci 2000;45(7): 1405-12
Saito N, Sato F, Kato M, Takeda H, Sugiyama T, Asaka M. Detection of coccoid Helicobacter pylori: light microscopical immunogold silver enhancing stain. Helicobacter. 1998;3(3):170-3
Snyder J D, Veldhuyzen Van Zanten S. Novel diagnostic tests to detect Helicobacter pylori infection: a pediatric perspective. Can J Gastroenterol 1999;13:585-9.
Song Q, Haller B, Schmid R, Adler G, Bode G. Helicobacter pylori in dental plaque, a comparison of different PCR primer sets. Dig Dis Sci 1999;44:479-84
Veldhuyzen Van Zanten S, Tytgat K M, deGara C J, Goldie J, Rashid F A, Bowen B M, et al. A prospective comparison of symptoms and five diagnostic tests in patients with Helicobacter pylori positive and negative dyspepsia. Eur J Gastroenterol Hepatol 1991;3:463-8.

1. A method of detecting a bacteria in a specimen, the method comprising:
(a) performing a multiplex polymerase chain reaction, wherein a plurality of nucleic acid fragments representing a plurality of loci in the bacteria are amplified by a plurality of forward and reverse primers, wherein for each pair of forward and reverse primer, a primer is internal; and
(b) determining that a specimen is positive for the bacteria if a number of amplified nucleic acid fragments is sufficient to detect the bacteria in the specimen.

2. The method of claim 1, wherein the bacteria is Helicobacter pylori.

3. The method of claim 1, wherein the plurality of loci is selected from the group consisting of nucleic acid regions whose sequences are designated a 0.86 kb DNA fragment, Urea A gene, 16S rRNA, a DNA sequence encoding a 26 kDa antigen, Hpa A gene, CagA, UreaC, and Flagellin.

4. The method of claim 1, wherein the plurality of primers is selected from the group consisting of primers whose DNA sequences listed in TABLE 2.

5. The method of claim 1, wherein the primers comprise the following characteristics:
(a) about twenty to thirty bases long;
(b) melting temperature of about 60oC;
(c) GC content of about fifty percent;
(d) minimal dimer formation; and
(e) low frequency of mutations in the primer binding site.

6. A method for detecting Helicobacter pylori in a specimen, the method comprising:
(a) performing a multiplex polymerase chain reaction, wherein a plurality of primers amplify a plurality of Helicobacter pylori nucleic acid fragments are selected from the group consisting of nucleic acid molecules whose sequences are designated 0.86 kb DNA fragment, Urea A gene, 16S rRNA, a DNA sequence encoding a 26 kDa antigen, Hpa A gene, CagA, UreaC, and Flagellin; and
(b) determining that a specimen is positive for Helicobacter pylori if a number of amplified fragments is sufficient to detect the bacteria in the specimen.

7. The method of claim 6, wherein the plurality of nucleic acid fragments comprise a plurality of internal fragments.

8. The method of claim 6, wherein the multiplex polymerase chain reaction is performed in a single reaction chamber.

9. The method of claim 6, wherein the plurality of Helicobacter pylori nucleic acid fragments is selected from the group consisting of RNA, cDNA, and genomic DNA.

10. The method of claim 6, wherein the plurality of Helicobacter pylori nucleic acid fragments is selected from the group consisting of coding, non-coding, exon, intron, and regulatory region.

11. The method of claim 6, wherein the plurality of primer pairs are derived from the group consisting of nucleic acid molecules whose sequences are designated 0.86 kb DNA fragment, Urea A gene, 16S rRNA, a DNA sequence encoding a 26 kDa antigen, Hpa A gene, CagA, UreaC, and Flagellin.

12. The method of claim 6, wherein the multiplex polymerase chain reaction is performed with an isolated nucleic acid.

13. The method of claim 6, wherein the multiplex polymerase chain reaction is performed directly with a biological sample selected from the group consisting of cell culture, bacterial cell culture, gastrointestinal tract tissue, stool, urine, blood, saliva, mucus secretions, dental plaque, and other sample capable of containing H. pylori.

14. The method of claim 12, wherein the bacterial DNA is isolated from a clinical sample.

15. The method of claim 14, wherein the clinical sample is selected from the group consisting of gastrointestinal tract tissue, stool, urine, blood, saliva, mucus secretions, dental plaque, and other tissues capable of containing Helicobacter pylori.

16. The method of claim 6, wherein the primer pairs are selected from the primers listed in TABLE 2.

17. The method of claim 6 further comprising performing a multiplex polymerase chain reaction, wherein up to ten DNA fragments representing five Helicobacter pylori loci are amplified by fifteen primers, the ten DNA fragments representing five internal fragments.

18. The method of claim 17, wherein the fifteen primers comprise five forward and ten reverse primers.

19. The method of claim 6, wherein the specimen is considered positive if at least fifty percent of all of the plurality of nucleic acid fragments or at least four DNA fragments representing two loci in Helicobacter pylori are amplified.

20. A method of detecting Helicobacter pylori in a specimen, the method comprising:
(a) designing a plurality of primers in a plurality loci of Helicobacter pylori, wherein the primers are
(i) specific for Helicobacter pylori;
(ii) have a low frequency of mutations in primer binding sites;
(b) performing a multiplex polymerase chain reaction wherein the plurality of primers amplify a plurality of nucleic acid fragments representing the plurality of loci in Helicobacter pylori; and
(c) determining that a specimen is positive for Helicobacter pylori if at least fifty percent of all the nucleic acid fragments or at least four DNA fragments representing two loci in Helicobacter pylori are amplified.

21. A diagnostic kit to detect H. pylori in a specimen comprising:
(a) a plurality of primers to amplify a plurality of nucleic acid fragments representing a plurality of loci in H. pylori; and
(b) reagents to perform a multiplex polymerase chain reaction.

22. The diagnostic kit of claim 21, wherein the plurality of primers comprise a first set of primers to amplify a first set of nucleic acid fragments, a second set of primers to amplify a second set of nucleic acid fragments that are internal to the first set of DNA fragments.

23. The diagnostic kit of claim 21, wherein the reagents comprise a DNA polymerase, nucleotides, and buffers.

24. The diagnostic kit of claim 22, wherein the first and second set of primers comprise a plurality of DNA molecules comprising nucleotide sequences listed in TABLE 2.

25. The diagnostic kit of claim 21, wherein the reagents comprise:
(a) a buffer comprising 10 mM Tris-HCl (pH 8.3), 50 mM KCl, 1.6 mM MgCl2, 0.001% (weight/volume) gelatin; and
(b) 0.3 mM of each deoxynuclotide.

26. The diagnostic kit of claim 21, wherein the primers have a concentration of about 0.05 μM.

27. A primer from one of five loci in H. pylori, the primer selected from the group consisting of nucleic acid molecules with sequences

| | |
|---|---|
| AACGC CGTGA GTTCG TCGTA TCG | (SEQ ID NO:1) |
| CATAT AGCCG CTTTT TCTGG TGTCT TTA | (SEQ ID NO:2) |
| CCTCA CGCCA TCAGT CCCAA AAAT | (SEQ ID NO:3 |
| TGATA GGCAA GCAGA CAACG AA | (SEQ ID NO:4) |
| GATGT GTGTG TCAAT ACCAC CAGC | (SEQ ID NO:5) |
| GCAGG ACCCA CGCTA AGATT GT, | (SEQ ID NO:6) |
| CAGGT CGCCT TCGCA ATGAG TA, | (SEQ ID NO:7) |
| ACGGG AGGCA GCAGT AGGGA ATA, | (SEQ ID NO:8) |
| GCCGC GTGGA GGATG AAGGT, | (SEQ ID NO:9) |
| TCATG CCTTT ATCGC CTTTT CTCC, | (SEQ ID NO:10) |
| GTGGA AAAAG GCGGT ATCGG TCAA, | (SEQ ID NO:11) |
| CGATC GCTTT GAGAG GTGCT TTTT, | (SEQ ID NO:12) |
| CTAGA GCCTA TGAGT GGGGA ATCTT T, | (SEQ ID NO:13) |
| ATCCG TTCCC TTAAC CATAG TGCT, | (SEQ ID NO:14) |
| and | |
| TGCTA ACTAA CCCCC CGCTA TGGC. | (SEQ ID NO:15) |
